SEQUENCE LISTING (35 sequences)

SEQ ID NO: 1 - RNA, 459 nt, Homo sapiens
  source 1..459: mol_type = mRNA
gtgtcaccaccagagaaggatgatggccagaaaggtgaagaagctgtcggtagcacagagcatcctgaggaagtcacaacccaagcggaagctgcaattgaagagggggaggtggagacagaaggggaagcagcagtggaaggggaagaggaggctgtgtcctatggagatgctgaaagcgaagaggaatattactatacagaaacttcatccccggaagggcaaatcagtgctgcagatacgacttacccgtatttcagtcctcctcaggaactgcctggagaggaggcatacgatagtgttagcggggaggctggtctccaaggcttccagcaagaggccaccggtccaccagaatccagagaaaggagggtcacctccccagagccatcccacggagtcttaggcccgtcggagcaaatgggccaggtcacctctgggccagcagtgggcagattg

SEQ ID NO: 2 - RNA, 29 nt, Homo sapiens
  source 1..29: mol_type = mRNA
tgtcaccaccagagaaggatgatggccag

SEQ ID NO: 3 - RNA, 28 nt, Homo sapiens
  source 1..28: mol_type = mRNA
aggatgatggccagaaaggtgaagaagc

SEQ ID NO: 4 - RNA, 28 nt, Homo sapiens
  source 1..28: mol_type = mRNA
aaggtgaagaagctgtcggtagcacaga

SEQ ID NO: 5 - RNA, 20 nt, Synthetic construct
  source 1..20: mol_type = other RNA
catcatccttctctggtggt

SEQ ID NO: 6 - RNA, 20 nt, Synthetic construct
  source 1..20: mol_type = other RNA
cttcacctttctggccatca

SEQ ID NO: 7 - RNA, 20 nt, Synthetic construct
  source 1..20: mol_type = other RNA
tgctaccgacagcttcttca

SEQ ID NO: 8 - RNA, 49 nt, Homo sapiens
  source 1..49: mol_type = mRNA
accaccagagaaggatgatggccagaaaggtgaagaagctgtcggtagc

SEQ ID NO: 9 - RNA, 20 nt, Synthetic construct
  source 1..20: mol_type = other RNA
gtggtgacacctgtaaaggt

SEQ ID NO: 10 - RNA, 20 nt, Synthetic construct
  source 1..20: mol_type = other RNA
cctcaggatgctctgtgcta

SEQ ID NO: 11 - RNA, 20 nt, Synthetic construct
  source 1..20: mol_type = other RNA
cttgggttgtgacttcctca

SEQ ID NO: 12 - RNA, 20 nt, Synthetic construct
  source 1..20: mol_type = other RNA
caattgcagcttccgcttgg

SEQ ID NO: 13 - RNA, 20 nt, Synthetic construct
  source 1..20: mol_type = other RNA
ccacctccccctcttcaatt

SEQ ID NO: 14 - RNA, 20 nt, Synthetic construct
  source 1..20: mol_type = other RNA
cccttctgtctccacctccc

SEQ ID NO: 15 - RNA, 20 nt, Synthetic construct
  source 1..20: mol_type = other RNA
cttccccttctgtctccacc

SEQ ID NO: 16 - RNA, 20 nt, Synthetic construct
  source 1..20: mol_type = other RNA
cccttccactgctgcttccc

SEQ ID NO: 17 - RNA, 20 nt, Synthetic construct
  source 1..20: mol_type = other RNA
ccccttccactgctgcttcc

SEQ ID NO: 18 - RNA, 20 nt, Synthetic construct
  source 1..20: mol_type = other RNA
acacagcctcctcttcccct

SEQ ID NO: 19 - RNA, 20 nt, Synthetic construct
  source 1..20: mol_type = other RNA
actgatttgcccttccgggg

SEQ ID NO: 20 - RNA, 20 nt, Synthetic construct
  source 1..20: mol_type = other RNA
cctcctctccaggcagttcc

SEQ ID NO: 21 - RNA, 20 nt, Synthetic construct
  source 1..20: mol_type = other RNA
ggctactcaccaatctgccc

SEQ ID NO: 22 - RNA, 20 nt, Synthetic construct
  source 1..20: mol_type = other RNA
ctctggtggtgacacctgta

SEQ ID NO: 23 - RNA, 20 nt, Synthetic construct
  source 1..20: mol_type = other RNA
cttctctggtggtgacacct

SEQ ID NO: 24 - RNA, 20 nt, Synthetic construct
  source 1..20: mol_type = other RNA
atccttctctggtggtgaca

SEQ ID NO: 25 - RNA, 20 nt, Synthetic construct
  source 1..20: mol_type = other RNA
atcatccttctctggtggtg

SEQ ID NO: 26 - RNA, 20 nt, Synthetic construct
  source 1..20: mol_type = other RNA
gccatcatccttctctggtg

SEQ ID NO: 27 - RNA, 20 nt, Synthetic construct
  source 1..20: mol_type = other RNA
ctggccatcatccttctctg

SEQ ID NO: 28 - RNA, 20 nt, Synthetic construct
  source 1..20: mol_type = other RNA
tttctggccatcatccttct

SEQ ID NO: 29 - RNA, 20 nt, Synthetic construct
  source 1..20: mol_type = other RNA
acctttctggccatcatcct

SEQ ID NO: 30 - RNA, 20 nt, Synthetic construct
  source 1..20: mol_type = other RNA
ttcacctttctggccatcat

SEQ ID NO: 31 - RNA, 20 nt, Synthetic construct
  source 1..20: mol_type = other RNA
ttcttcacctttctggccat

SEQ ID NO: 32 - RNA, 20 nt, Synthetic construct
  source 1..20: mol_type = other RNA
agcttcttcacctttctggc

SEQ ID NO: 33 - RNA, 20 nt, Synthetic construct
  source 1..20: mol_type = other RNA
gacagcttcttcacctttct

SEQ ID NO: 34 - RNA, 20 nt, Synthetic construct
  source 1..20: mol_type = other RNA
accgacagcttcttcacctt

SEQ ID NO: 35 - RNA, 20 nt, Synthetic construct
  source 1..20: mol_type = other RNA
gctaccgacagcttcttcac

(19) United States
(12) Patent Application Publication
(10) Pub. No.: US 2026/0117228 A1
(43) Pub. Date: Apr. 30, 2026

(54) COMPOSITIONS AND METHODS FOR TREATING PRIMARY CILIARY DYSKINESIA

(71) Applicant: SKIP THERAPEUTICS LTD., Ness Ziona (IL)

(72) Inventors: Ariel FEIGLIN, Mazkeret Batya (IL); Yehezkel SZTAINBERG, Rehovot (IL); Ilana BUCHUMENSKI, Ashkelon (IL); Maya David TEITELBAUM, Rishon LeTsiyon (IL); Hagit T. PORATH, Moshav Gimzo (IL)

(21) Appl. No.: 19/006,435

(22) Filed: Dec. 31, 2024

Related U.S. Application Data
(63) Continuation of application No. PCT/IL2023/050702, filed on Jul. 6, 2023.
(60) Provisional application No. 63/358,951, filed on Jul. 7, 2022.

(51) Int. Cl. C12N 15/113

(52) U.S. Cl. CPC C12N 15/113; C12N 2310/11; C12N 2310/315; C12N 2310/321; C12N 2320/33

(57) ABSTRACT

The present invention is directed to a method for treating primary ciliary dyskinesia (PCD) using a splicing modulator, such as an antisense oligonucleotide, capable of inducing the skipping of exon 3 of the coiled-coil domain containing 40 (CCDC40) pre-mRNA. Also provided is a composition comprising the splicing modulator, and use of same.

### CROSS-REFERENCE TO RELATED-APPLICATION

This application is a ByPass Continuation of PCT Patent Application No. PCT/IL2023/050702 having International filing date of Jul. 6, 2023, which claims the benefit of priority of U.S. Provisional Patent Application No. 63/358,951, titled “COMPOSITIONS AND METHODS FOR TREATING PRIMARY CILIARY DYSKINESIA”, filed Jul. 7, 2022, the contents of which are all incorporated herein by reference in their entirety.

### REFERENCE TO AN ELECTRONIC SEQUENCE LISTING

The contents of the electronic sequence listing (SKIP-P-001-PCT ST26.xml; size: 32,229 bytes; and date of creation: Jun. 14, 2023) is herein incorporated by reference in its entirety.

### FIELD OF INVENTION

The present invention is in the field of antisense oligonucleotides and therapeutic use of the antisense oligonucleotides.

### BACKGROUND

Primary ciliary dyskinesia (PCD) is an autosomal recessive disorder of cilia structure, function, and biogenesis leading to chronic infections of the respiratory tract, fertility problems and disorders of organ laterality. PCD can be caused by mutations in multiple genes. Specifically, mutations in the Coiled-Coil Domain Containing 39 and 40 (CCDC39 and CCDC40) genes have been shown to be a major cause of PCD. The CCDC39 and CCDC40 genes encode structurally related coiled-coil domain-containing proteins, which play an important role in cilia formation. Cilia are microtubule-based, hair-like organelles, that extend from the surface of multiple cell types of the human body, involved in motile and sensory functions. There are two major types of cilia: non-motile and motile cilia. Non-motile cilia serve as sensory organelles while motile cilia provide movement, as in sperm and in the lungs and trachea of the respiratory tract. CCDC39 and CCDC40 are crucial for normal function of the motile cilia.

Functional restoration of gene function in genetic disorders has recently been achieved by modulating splicing of the mutated genes using antisense oligonucleotides (ASOs). ASOs are single stranded, chemically modified, nucleic acids that bind pre-mRNA of the target gene and alter splicing in a way that can restore gene functionality. For example, a nonsense mutation causing early truncation of a protein can be rescued by using an ASO to induce exclusion of the mutated exon (also known as exon skipping) and thereby preventing early termination of the protein. This will lead to formation of a shorter protein than the natural protein but still may be fully or partially functional. Moreover, nonsense mutations often trigger RNA degradation via the nonsense mediated decay (NMD) pathway and therefore result in no functional protein from the mutated allele. Therefore, the skipped protein replaces a situation of no protein at all. Using this strategy, ASO targeting of exon 23 in CFTR have shown encouraging in-vitro results for treatment of nonsense mutations in Cystic Fibrosis. Similarly, it is possible to correct frameshift mutations by inducing exclusion of an exon of the correct length to restore the original reading frame up and downstream of the mutation. For example, multiple ASO treatments have been approved for treatment of Duchene Muscular Dystrophy targeting exons that restore the original reading frame for some mutations.

There is still a great need for ASOs specifically inducing exon 3 skipping of the CCDC40 pre-mRNA, and methods of using same, such as for treating or ameliorating PCD.

### SUMMARY

According to one aspect, there is provided a method for treating primary ciliary dyskinesia (PCD) in a subject in need thereof, comprising administering to the subject a therapeutically effective amount of at least one synthetic antisense oligonucleotide (ASO), wherein the ASO induces the skipping of exon 3 of the coiled-coil domain containing 40 (CCDC40) pre-mRNA, thereby treating PCD in the subject.

According to another aspect, there is provided a composition comprising an ASO comprising 14 to 25 bases having at least 80% complementarity to a CCDC40 pre-mRNA and characterized by inducing splicing activity of exon 3 of the CCDC40 pre-mRNA.

In some embodiments, the ASO comprises a backbone selected from the group consisting of: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

In some embodiments, the ASO comprises 14 to 25 bases.

In some embodiments, the ASO has at least 75% complementarity to an equal-length portion of a nucleic acid sequence derived from the polynucleotide sequence:

(SEQ ID NO: 1)
GTGTCACCACCAGAGAAGGATGATGGCCAGAAAGGTGAAGAAGCT
GTCGGTAGCACAGAGCATCCTGAGGAAGTCACAACCCAAGCGGAA
GCTGCAATTGAAGAGGGGGAGGTGGAGACAGAAGGGGAAGCAGCA
GTGGAAGGGGAAGAGGAGGCTGTGTCCTATGGAGATGCTGAAAGC
GAAGAGGAATATTACTATACAGAAACTTCATCCCCGGAAGGGCAA
ATCAGTGCTGCAGATACGACTTACCCGTATTTCAGTCCTCCTCAG
GAACTGCCTGGAGAGGAGGCATACGATAGTGTTAGCGGGGAGGCT
GGTCTCCAAGGCTTCCAGCAAGAGGCCACCGGTCCACCAGAATCC
AGAGAAAGGAGGGTCACCTCCCCAGAGCCATCCCACGGAGTCTTA
GGCCCGTCGGAGCAAATGGGCCAGGTCACCTCTGGGCCAGCAGTG
GGCAGATTG.

In some embodiments, the ASO has at least 75% complementarity to any one of:

(SEQ ID NO: 2)
TGTCACCACCAGAGAAGGATGATGGCCAG,
(SEQ ID NO: 3)
AGGATGATGGCCAGAAAGGTGAAGAAGC,
and
(SEQ ID NO: 4)
AAGGTGAAGAAGCTGTCGGTAGCACAGA.

In some embodiments, the ASO comprises the nucleic acid sequence set forth in SED ID Nos: 5-7, 23-26, and 28-35.

In some embodiments, the ASO comprises the nucleic acid sequence set forth in SED ID Nos: 5, 6, 23, 25, 26, and 30-33.

In some embodiments, the ASO comprises the nucleic acid sequence set forth in SED ID Nos: 5, 23, 25, 26, 30, and 32.

In some embodiments, the subject comprises at least one nonsense and/or frameshift mutation in exon 3 of CCDC40.

In some embodiments, the at least one mutation is c.248delC.

In some embodiments, the ASO comprises the nucleic acid sequence of:

(SEQ ID NO: 5)
CATCATCCTTCTCTGGTGGT;
(SEQ ID NO: 6)
CTTCACCTTTCTGGCCATCA;
or
(SEQ ID NO: 7)
TGCTACCGACAGCTTCTTCA

In some embodiments, the ASO comprises a chemically modified backbone.

In some embodiments, the chemically modified backbone comprises: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

In some embodiments, the composition further comprises a pharmaceutically acceptable carrier.

In some embodiments, the composition is for use in inducing the skipping of exon 3 of the CCDC40 pre-mRNA in a subject in need thereof, or a cell of the subject.

In some embodiments, the subject is afflicted with PCD.

In some embodiments, the composition is an inhalation composition.

In some embodiments, the composition is for use in the treatment of PCD in a subject in need thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fec.

FIGS. 1A-1F include a non-limiting scheme, micrographs, and vertical bar graphs showing antisense oligonucleotide mini screen for exon 3 skipping in CCDC40. (1A) Design of CCDC40 plasmids, CCDC40 wild-type (CCDC40^{WT}) and CCDC40 with exon 3 skipped (CCDC40^{ex3skp}). (1B) Western blot analysis of protein expression from HEK293 cells 48 hrs. following transfection with CCDC40^{WT} and CCDC40^{ex3skp} plasmids. NT—not transfected. GAPDH is a housekeeping protein used to normalize for total protein in the lysate. (1C) Densitometric quantification of the western blot in (1B). (1D) Oligo-screen including 30 splice switching antisense oligonucleotides (first screen: ASOs 1-16 left panel and second screen: ASOs 17-30-right panel) to induce skipping (exclusion) of CCDC40 exon 3 in A549 lung epithelium cells. As ASO2 was identified as a promising and efficient ASO, a second screen (ASOs 17-30) was further designed so as to target the sequence close to the binding region of ASO2 at a higher resolution. Ctrl-antisense control. GAPDH is a housekeeping gene used for normalization. (1E) Densitometric quantifications (ASOs 1-16 left panel and 17-30-right panel) of the lower band (CCDC40^{ex3skp}) in (1D). (1F) A snapshot image from the UCSC browser showing the genomic position of ASOs 1-30 from (1D), spanning the region of exon 3 of CCDC40.

FIG. 2 includes fluorescent micrographs showing that CCDC40^{ex3skp} co-localizes with microtubules. Immunofluorescence analysis of CCDC40^{WT} and CCDC40^{ex3skp} co-localization with microtubules in Hela cells after transient transfection of plasmids (48 hours). CCDC40 protein is tagged in its C-terminus with Flag and detected with an anti-Flag antibody. Microtubule are detected using an anti-Tubulin specific antibody. Cells' nuclei are stained with Hoechst 33342.

FIGS. 3A-3D include micrographs, a vertical bar graph, and non-limiting schemes of design, showing that the CCDC40^{ex3skp} protein interacts with CCDC39 similarly to CCDC40^{WT} and that ASO-mediated exon 3 skipping can partially restore this interaction that is lost in CCDC40^{c.248delC} mutant. (3A) Western blot analysis of CCDC40 co-immunoprecipitation with CCDC39 in HEK293 cells 48 hrs. following transfection. Samples (transfection) included: (1) CCDC39-HA; (2) CCDC40^{WT}-Flag; (3) CCDC39-HA+CCDC40^{WT}-Flag; (4) CCDC39-HA+CCDC40^{SKP}-Flag; and (5) CCDC39-HA+LDLR-Flag. CCDC40 protein is tagged in its C-terminus with Flag and detected with an anti-Flag antibody. CCDC39 protein is tagged in its C-terminus with HA and detected with an anti-HA antibody. Flag-tagged LDLR was used as a negative control (as it is not known to bind CCDC39). Input-refers to the total cell lysate before the immunoprecipitation assay. Co-IP—refers to the protein fraction eluted from the HA magnetic beads following incubation with the lysate. Unbound-refers to the protein fraction not bound to the HA magnetic beads following incubation with the lysate. (3B) Densitometric quantification of the western blot in (3A). (3C) Non-limiting design of flag-tagged CCDC40 plasmids with introns, CCDC40 wild-type (CCDC40^{WT}) and CCDC40 with the c.248delC mutation (CCDC40^{c.248deIC}). (3D) Western blot analysis of CCDC40 co-immunoprecipitation with CCDC39 in HEK293 cells 48 hrs. following transfection and ASO treatment. Samples (transfection) included: (1) CCDC39-HA+CCDC40^{WT}-Flag; (2) CCDC39-HA+CCDC40^{c.248delC}-Flag+ASO-ctrl 500 nM; (3) CCDC39-HA+CCDC40^{c.248delC}-Flag+ASO-2 500 nM; and (4) CCDC39-HA+CCDC40^{c.248delC}-Flag+ASO-2 100 nM.

FIGS. 4A-4B include fluorescent micrographs showing co-localization of CCDC40^{ex3skp} with CCDC39. (4A) Immunofluorescence analysis of CCDC39, CCDC40^{WT}, CCDC40^{ex3skp}, and Granulin (GRN, as a negative control as it is not known to interact with CCDC39) 48 hrs following transient transfection of each plasmid in different Hela cells cultures. CCDC40^{WT}, CCDC40^{ex3skp}, and Granulin are tagged in their C-terminus with Flag and detected with an anti-Flag antibody. CCDC39 is tagged in its C-terminus with HA and detected with an anti-HA antibody. Cells' nuclei are stained with Hoechst 33342. (4B) Immunofluorescence analysis of CCDC39, CCDC40^{WT}, CCDC40^{ex3skp}, and Granulin (GRN, as a negative control as it is not known to interact with CCDC39) 48 hrs. following transient co-transfections.

### DETAILED DESCRIPTION

### Method of Treatment

According to some embodiments, a method for treating primary ciliary dyskinesia (PCD) in a subject is provided. In some embodiments, the method comprises administering to the subject a therapeutically effective amount of a splicing modulator, wherein the splicing modulator induces the skipping of exon 3 of the coiled-coil domain containing 40 (CCDC40) pre-mRNA, thereby treating PCD in the subject.

In some embodiments, the method comprises administering a splicing modulator which is at least one synthetic antisense oligonucleotide (ASO).

In some embodiments, the ASO is an RNA ASO. In some embodiments, the ASO comprises RNA nucleobases.

Nucleic acid sequences as provided herein, in the context of ASOs of the invention, in some embodiments, comprise RNA nucleic acid sequences. In the context of ASOs as provided herein, as persons skilled in the art are aware, thymine is substituted with uracil, and is the base that is considered to be complementary to adenosine.

In some embodiments, the ASO is chemically modified. In some embodiments, the chemical modification is a modification of a backbone of the ASO. In some embodiments, the chemical modification is a modification of a sugar of the ASO. In some embodiments, the chemical modification is a modification of a nucleobase of the ASO. In some embodiments, the chemical modification increases stability of the ASO in a cell. In some embodiments, the chemical modification increases stability of the ASO in vivo. In some embodiments, the chemical modification increases the ASO's ability to modulate splicing. In some embodiments, the chemical modification increases the ASO's ability to induce skipping of exon 3 of the CCDC40 pre-mRNA. In some embodiments, the chemical modification increases the half-life of the ASO. In some embodiments, the chemical modification inhibits polymerase extension from the 3′ end of the ASO. In some embodiments, the chemical modification inhibits recognition of the ASO by a polymerase. In some embodiments, the chemical modification inhibits double-strand triggered degradation. In some embodiments, the chemically modified ASO does not trigger nucleic acid double-stranded degradation upon binding a CCDC40 pre-mRNA. In some embodiments, the chemical modification inhibits RISC-mediated degradation. In some embodiments, the chemical modification inhibits RISC-mediated degradation or any parallel nucleic acid degradation pathway.

In some embodiments, the ASO is devoid of a labeling moiety. In some embodiments, the ASO is not labeled. In some embodiments, the ASO does not emit a detectable signal or does not comprise moieties capable of being recognized so as to enable nucleic acid detection (e.g., digoxigenin and fluorescently labeled anti-DIG antibody). In some embodiments, a detectable signal comprises a dye or an emitting energy which provides detection of a compound, e.g., a polynucleotide, in vivo or in vitro. In some embodiments, a detectable signal comprises: a fluorescent signal, a chromatic signal, or a radioactive signal.

In some embodiments, the ASO is devoid of radioactive nucleobase(s); digoxigenin, streptavidin, biotin, a fluorophore, hapten label, CLICK label, amine label, or thiol label.

In some embodiments, the chemical modification is selected from: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, or any combination thereof.

In some embodiments, the ASO comprises at least 14 bases, at least 15 bases, at least 16 bases, at least 17 bases, at least 18 bases, at least 19 bases, at least 20 bases, at least 21 bases, at least 22 bases, at least 23 bases, at least 24 bases, or at least 25 bases, or any value and range therebetween. Each possibility represents a separate embodiment of the invention.

In some embodiments, the ASO comprises 14 to 30 bases, 14 to 28 bases, 14 to 26 bases, 14 to 24 bases, 14 to 21 bases, 14 to 19 bases, 14 to 18 bases, or 14 to 17 bases. Each possibility represents a separate embodiment of the invention. In some embodiments, the ASO comprises 17 to 22 bases.

In some embodiments, the ASO is complementary to exon 3 of the CDDC40 pre-mRNA. In some embodiments, exon 3 of the CCDC40 pre-mRNA comprises the sequence:

(SEQ ID NO: 1)
GTGTCACCACCAGAGAAGGATGATGGCCAGAAAGGTGAAGAAGCT
GTCGGTAGCACAGAGCATCCTGAGGAAGTCACAACCCAAGCGGAA
GCTGCAATTGAAGAGGGGGAGGTGGAGACAGAAGGGGAAGCAGCA
GTGGAAGGGGAAGAGGAGGCTGTGTCCTATGGAGATGCTGAAAGC
GAAGAGGAATATTACTATACAGAAACTTCATCCCCGGAAGGGCAA
ATCAGTGCTGCAGATACGACTTACCCGTATTTCAGTCCTCCTCAG
GAACTGCCTGGAGAGGAGGCATACGATAGTGTTAGCGGGGAGGCT
GGTCTCCAAGGCTTCCAGCAAGAGGCCACCGGTCCACCAGAATCC
AGAGAAAGGAGGGTCACCTCCCCAGAGCCATCCCACGGAGTCTTA
GGCCCGTCGGAGCAAATGGGCCAGGTCACCTCTGGGCCAGCAGTG
GGCAGATTG.

In some embodiments, the ASO is complementary to an equal-length portion of a nucleic acid sequence derived from the polynucleotide sequence comprising or consisting of:

(SEQ ID NO: 1)
GTGTCACCACCAGAGAAGGATGATGGCCAGAAAGGTGAAGAAGCT
GTCGGTAGCACAGAGCATCCTGAGGAAGTCACAACCCAAGCGGAA
GCTGCAATTGAAGAGGGGGAGGTGGAGACAGAAGGGGAAGCAGCA
GTGGAAGGGGAAGAGGAGGCTGTGTCCTATGGAGATGCTGAAAGC
GAAGAGGAATATTACTATACAGAAACTTCATCCCCGGAAGGGCAA
ATCAGTGCTGCAGATACGACTTACCCGTATTTCAGTCCTCCTCAG
GAACTGCCTGGAGAGGAGGCATACGATAGTGTTAGCGGGGAGGCT
GGTCTCCAAGGCTTCCAGCAAGAGGCCACCGGTCCACCAGAATCC
AGAGAAAGGAGGGTCACCTCCCCAGAGCCATCCCACGGAGTCTTA
GGCCCGTCGGAGCAAATGGGCCAGGTCACCTCTGGGCCAGCAGTG
GGCAGATTG.

In some embodiments, the ASO is complementary to an equal-length portion of a nucleic acid sequence derived from the polynucleotide sequence comprising or consisting of: ACCACCAGAGAAGGATGATGGCCAGAAAGGTGAAGAAGCTGTCGGTAGC (SEQ ID NO: 8).

In some embodiments, the ASO is complementary to any one of:

(SEQ ID NO: 2)
TGTCACCACCAGAGAAGGATGATGGCCAG,
(SEQ ID NO: 3)
AGGATGATGGCCAGAAAGGTGAAGAAGC,
and
(SEQ ID NO: 4)
AAGGTGAAGAAGCTGTCGGTAGCACAGA.

In some embodiments, the ASO is complementary to an equal-length portion of a nucleic acid sequence derived from the polynucleotide sequence comprising or consisting of any one of SEQ ID Nos: 1-4.

In some embodiments, the ASO has at least 75%, at least 80%, at least 85%, at least 90%, at least 95%, at least 99%, or 100% complementarity to SEQ ID Nos: 1-4, any value and range therebetween. Each possibility represents a separate embodiment of the invention. In some embodiments, the ASO has 70-80%, 75-85%, 80-90%, 85-95%, 90-99%, or 95-100% complementarity to SEQ ID Nos: 1-4. Each possibility represents a separate embodiment of the invention.

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

In some embodiments, the ASO comprises or consists of the nucleic acid sequence of: CATCATCCTTCTCTGGTGGT (SEQ ID NO: 5); CTTCACCTTTCTGGCCATCA (SEQ ID NO: 6); or TGCTACCGACAGCTTCTTCA (SEQ ID NO: 7).

In some embodiments, the ASO comprises or consists of a nucleic acid sequence selected from: GTGGTGACACCTGTAAAGGT (SEQ ID NO: 9); CCTCAGGATGCTCTGTGCTA (SEQ ID NO: 10); CTTGGGTTGTGACTTCCTCA (SEQ ID NO: 11); CAATTGCAGCTTCCGCTTGG (SEQ ID NO: 12); CCACCTCCCCCTCTTCAATT (SEQ ID NO: 13); CCCTTCTGTCTCCACCTCCC (SEQ ID NO: 14); CTTCCCCTTCTGTCTCCACC (SEQ ID NO: 15); CCCTTCCACTGCTGCTTCCC (SEQ ID NO: 16); CCCCTTCCACTGCTGCTTCC (SEQ ID NO: 17); ACACAGCCTCCTCTTCCCCT (SEQ ID NO: 18); ACTGATTTGCCCTTCCGGGG (SEQ ID NO: 19); CCTCCTCTCCAGGCAGTTCC (SEQ ID NO: 20); GGCTACTCACCAATCTGCCC (SEQ ID NO: 21); CTCTGGTGGTGACACCTGTA (SEQ ID NO: 22); CTTCTCTGGTGGTGACACCT (SEQ ID NO: 23); ATCCTTCTCTGGTGGTGACA (SEQ ID NO: 24); ATCATCCTTCTCTGGTGGTG (SEQ ID NO: 25); GCCATCATCCTTCTCTGGTG (SEQ ID NO: 26); CTGGCCATCATCCTTCTCTG (SEQ ID NO: 27); TTTCTGGCCATCATCCTTCT (SEQ ID NO: 28); ACCTTTCTGGCCATCATCCT (SEQ ID NO: 29); TTCACCTTTCTGGCCATCAT (SEQ ID NO: 30); TTCTTCACCTTTCTGGCCAT (SEQ ID NO: 31); AGCTTCTTCACCTTTCTGGC (SEQ ID NO: 32); GACAGCTTCTTCACCTTTCT (SEQ ID NO: 33); ACCGACAGCTTCTTCACCTT (SEQ ID NO: 34); or GCTACCGACAGCTTCTTCAC (SEQ ID NO: 35).

In some embodiments, the ASO comprises or consists of a nucleic acid sequence selected from: SED ID Nos: 5-7, 23-26, and 28-35.

In some embodiments, the ASO comprises or consists of a nucleic acid sequence selected from: SED ID Nos: 5, 6, 23, 25, 26, and 30-33.

In some embodiments, the ASO comprises or consists of a nucleic acid sequence selected from: SED ID Nos: 5, 23, 25, 26, 30, and 32.

TABLE 1
Internal reference of the numbering of ASOs of
the invention and their respective SEQ ID NO.

| ASO # as provided in the | SEQ ID |
|---|---|
| description (including FIGS.) | NO: |
| 1 | 9 |
| 2 | 5 |
| 3 | 6 |
| 4 | 7 |
| 5 | 10 |
| 6 | 11 |
| 7 | 12 |
| 8 | 13 |
| 9 | 14 |
| 10 | 15 |
| 11 | 16 |
| 12 | 17 |
| 13 | 18 |
| 14 | 19 |
| 15 | 20 |
| 16 | 21 |
| 17 | 22 |
| 18 | 23 |
| 19 | 24 |
| 20 | 25 |
| 21 | 26 |
| 22 | 27 |
| 23 | 28 |
| 24 | 29 |
| 25 | 30 |
| 26 | 31 |
| 27 | 32 |
| 28 | 33 |
| 29 | 34 |
| 30 | 35 |

In some embodiments, the pre-mRNA is a wildtype pre-mRNA. In some embodiments, the pre-mRNA is a mutated pre-mRNA. In some embodiments, the CCDC40 pre-mRNA comprises any one of: SEQ ID Nos: 1-4. In some embodiments, the ASO is complementary to a nucleic acid sequence comprising any one of: SEQ ID Nos: 1-4.

In some embodiments, the ASO comprises an active fragment of any one of SEQ ID Nos: 5-7.

As used herein, the term “active fragment” refers to a fragment that is 100% identical to a contiguous portion of the full nucleotide sequence of the ASO, providing that at least: 30%, 40%, 50%, 60%, 70%, 80% or 90% of the activity of the original ASO nucleotide sequence is retained, or any value and range therebetween. Each possibility represents a separate embodiment of the present invention.

In some embodiments, the ASO is specific to a CCDC40 pre-mRNA.

As used herein, the term “specific” refers to both base pair specificity and also gene specificity. In some embodiments, the ASO is specific to the CCDC40 gene. In some embodiments, the ASO is specific to a splice silencing motif in CCDC40. In some embodiments, the ASO is specific to a splice silencing region of CCDC40. In some embodiments, the splice silencing is splice-silencing of exon 3 of CCDC40.

In some embodiments, the ASO binds the CCDC40 pre-mRNA with perfect complementarity. In some embodiments, the ASO does not bind any gene or pre-mRNA product thereof, other than CCDC40 with perfect complementarity. In some embodiments, the ASO does not bind any gene or pre-mRNA product thereof, other than CCDC40 with a complementarity of greater than 70, 75, 80, 85, 90, 95, 97, 99 or 100%. Each possibility represents a separate embodiment of the invention. In some embodiments, the ASO does not bind any gene or pre-mRNA product thereof, other than CCDC40 with a complementarity of greater than 90%. In some embodiments, the ASO binds any one of: SEQ ID Nos: 1-4 with perfect complementarity. In some embodiments, the ASO does not bind any sequence other than SEQ ID Nos: 1-4 with complementarity of greater than 70, 75, 80, 85, 90, 95, 97, 99 or 100%. Each possibility represents a separate embodiment of the invention. In some embodiments, the ASO does not bind any sequence other than SEQ ID Nos: 1-4 with a complementarity of greater than 90%. In some embodiments, the ASO does not bind with perfect complementarity to anywhere in the genome or transcriptome (including pre-transcriptome, e.g., transcriptome comprising or consisting of pre-mRNA) of a cell other than within CCDC40. In some embodiments, the ASO does not bind with complementarity of greater than 70, 75, 80, 85, 90, 95, 97, 99 or 100% to anywhere in the genome or transcriptome (including pre-transcriptome, e.g., transcriptome comprising or consisting of pre-mRNA) of a cell other than within CCDC40. Each possibility represents a separate embodiment of the invention. In some embodiments, the cell is a mammalian cell. In some embodiments, the mammal is a human.

In some embodiments, the ASO modulates expression of CCDC40. In some embodiments, the ASO modulates splicing of CCDC40. In some embodiments, the ASO modulates splicing of exon 3 of CCDC40. In some embodiments, the ASO does not cause an off-target effect. In some embodiments, off-target is a target other than CCDC40. In some embodiments, off-target is a target other than splicing of exon 3 of CCDC40. In some embodiments, the ASO does not substantially or significantly modulate expression of a gene other than CCDC40. In some embodiments, the ASO does not substantially or significantly modulate splicing of a gene other than CCDC40. In some embodiments, the ASO does not substantially or significantly modulate splicing of an exon other than exon 3 of CCDC40. In some embodiments, substantial modulation of expression is a change in expression of at least 5, 10, 15, 20, 25, 30, 35, 40, 45 or 50%. Each possibility represents a separate embodiment of the invention. In some embodiments, substantial modulation of expression is a change in expression of at least 20%.

In some embodiments, the ASO is complementary to an exon-intron junction. In some embodiments the exon is exon 3 of the CCDC40 pre-mRNA. In some embodiments, the ASO is complementary to an exon 3-intron 2 junction (or intron 2-exon 3) of the CCDC40 pre-mRNA. In some embodiments, the ASO is complementary to an exon 3-intron 3 junction of the CCDC40 pre-mRNA.

As used herein, an exon-intron junction comprising a portion of or all of exon 3 or may be referred to as exon 3-intron junction. In some embodiments, an exon 3-intron junction comprises the 5′ prime end of exon 3. In some embodiments, an exon 3-intron junction comprises the 3′ prime end of exon 3. In some embodiments, an exon 3-intron junction comprises the complete sequence of exon 3. In some embodiments, an exon 3-intron junction comprises the 3′ prime end of intro 2. In some embodiments, an exon 3-intron junction comprises the 5′ prime end of intron 3.

In some embodiments, the ASO is at least 70%, at least 80%, at least 85%, at least 90%, at least 95%, at least 99%, or 100% complementary to an exon 3-intron junction of the CCDC40 pre-mRNA, or any value and range therebetween. Each possibility represents a separate embodiment of the invention. In some embodiments, the ASO is 70-85%, 80-90%, 85-95%, 90-99%, or 95-100% complementary to an exon 3-intron junction of the CCDC40 pre-mRNA. Each possibility represents a separate embodiment of the invention.

In some embodiments, an ASO as disclosed herein targets, complements, induces, or any combination thereof, the skipping of exon 3 of CCDC40 pre-mRNA transcribed from a mutated allele of the CCDC40 gene. In some embodiments, an ASO as disclosed herein does not target, complement, induce, or any combination thereof, the skipping of exon 3 of CCDC40 pre-mRNA transcribed from a wildtype allele of the CCDC40 gene.

In some embodiments, the subject comprises or is characterized by having a genome comprising at least one mutation in exon 3 of CCDC40 rendering a partially or fully non-functional CFTR protein.

In some embodiments, the nonsense and/or frameshift mutation.

In some embodiments, the at least one mutation is c.248delC.

In some embodiments, “a mutation” as used herein, refers to a nucleotide substitution or modification which induces or results in a “primary ciliary dyskinesia phenotype” in a subject harboring or comprising the mutation.

In some embodiments, a modification comprises insertion, deletion, inversion, or a combination thereof, as long as the modification results in a primary ciliary dyskinesia phenotype in a subject harboring or comprising the modification.

As used herein, the term “primary ciliary dyskinesia phenotype” encompasses any symptom or manifestation related to PCD. Methods for diagnosing PCD and/or symptoms associated therewith are common and would be apparent to one of ordinary skill in the art.

In some embodiments, the method is directed to improving at least one clinical parameter of PCD in the subject.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “condition” includes anatomic and physiological deviations from the normal that constitute an impairment of the normal state of the living animal or one of its parts, that interrupts or modifies the performance of the bodily functions.

As used herein, the terms “subject” or “individual” or “animal” or “patient” or “mammal,” refers to any subject, particularly a mammalian subject, for whom therapy is desired, for example, a human.

In some embodiments, there is provided a method for treating primary ciliary dyskinesia (PCD) in a subject in need thereof, comprising administering to the subject a therapeutically effective amount of a synthetic antisense oligonucleotide (ASO), wherein the ASO induces the skipping of exon 3 of the CCDC40 pre-mRNA, thereby treating PCD in the subject.

### Composition

According to some embodiments, there is provided a composition comprising an ASO comprising 14 to 30 bases having at least 80% complementarity to a CCDC40 pre-mRNA and characterized by inducing splicing activity of exon 3 of the CCDC40 pre-mRNA.

In some embodiments, the composition comprises a plurality of ASOs characterized by inducing splicing activity of different target pre-mRNA.

The term “plurality” as used herein refers to any integer being equal to or greater than 2.

In some embodiments, the composition further comprises a pharmaceutically acceptable carrier.

The term “pharmaceutically acceptable carrier” as used herein refers to any of the standard pharmaceutical carriers known in the field such as sterile solutions, tablets, coated tablets, and capsules. Typically, such carriers contain excipients such as starch, milk, sugar, certain types of clay, gelatin, stearic acids or salts thereof, magnesium or calcium stearate, talc, vegetable fats or oils, gums, glycols, or other known excipients. Such carriers may also include flavor and color additives or other ingredients. Examples of pharmaceutically acceptable carriers include, but are not limited to, the following: water, saline, buffers, inert, nontoxic solids (e.g., mannitol, talc). Compositions comprising such carriers are formulated by well-known conventional methods. Depending on the intended mode of administration and the intended use, the compositions may be in the form of solid, semi-solid, or liquid dosage forms, such, for example, as powders, granules, crystals, liquids, suspensions, liposomes, nano-particles, nano-emulsions, pastes, creams, salves, etc., and may be in unit-dosage forms suitable for administration of relatively precise dosages.

In some embodiments, the pharmaceutical composition is formulated for oral, administration. In some embodiments, the pharmaceutical composition is formulated for nasal administration. In some embodiments, the pharmaceutical composition is formulated for administration by inhalation. In some embodiments, the pharmaceutical composition is formulated for abdominal administration. In some embodiments, the pharmaceutical composition is formulated for subcutaneous administration. In some embodiments, the pharmaceutical composition is formulated for intra-peritoneal administration. In some embodiments, the pharmaceutical composition is formulated for intravenous administration.

In some embodiments, the pharmaceutical composition is formulated for systemic administration. In some embodiments, the pharmaceutical composition is formulated for administration to a subject. In some embodiments, the subject is a human subject. It will be understood by a skilled artisan that a pharmaceutical composition intended to administration to a subject should not have off-target effects, e.g., effects other than the intended therapeutic ones. In some embodiments, the pharmaceutical composition is devoid of a substantial effect on a gene other than CCDC40. In some embodiments, the pharmaceutical composition is devoid of a substantial effect on splicing of an exon other than exon 3 of CCDC40. In some embodiments, a substantial effect is one with a phenotypic result. In some embodiments, a substantial effect is a deleterious effect. In some embodiments, deleterious is with respect to the health and/or wellbeing of the subject.

In some embodiments, the composition is administered by inhalation. In some embodiments, the composition is an inhalation composition. In some embodiments, an ASO as disclosed and as described hereinabove, or a pharmaceutical composition comprising thereof, is used in the modulation of splicing of a CCDC40 pre-mRNA transcribed from a CCDC40 gene having a mutated exon 3.

The phrase “modulation of splicing” as used herein refers to affecting a change in the level of any RNA or mRNA variant produced by the CCDC40 native, mutated, or both, pre-mRNA.

In certain embodiments, the use is for reducing the level of an mRNA molecule comprising the mutated exon 3.

In some embodiments, an ASO as disclosed and as described hereinabove, or a pharmaceutical composition comprising thereof, is used in method for improving at least one clinical parameter of PCD. In some embodiments, an ASO as disclosed and as described hereinabove, or a pharmaceutical composition comprising thereof, is used in treating PCD.

### Method of Production

According to some embodiments, there is provided a method for producing a compound suitable for treating PCD.

In some embodiments, the method comprises obtaining a compound that binds to exon 3 of the CCDC40 pre-mRNA. In some embodiments, the method comprises assaying the skipping of exon 3 of the CCDC40 pre-mRNA in the presence of the obtained compound. In some embodiments, the method comprises selecting at least one compound that induces the exclusion of exon 3 from the CCDC40 pre-mRNA.

In some embodiments, the method comprises obtaining a compound that binds to exon 3 of the CCDC40 pre-mRNA, and assaying the skipping of exon 3 of the CCDC40 pre-mRNA in the presence of the obtained compound, and selecting at least one compound that induces the exclusion of exon 3 from the CCDC40 pre-mRNA, thereby producing a compound suitable for treating PCD.

In some embodiments, the method comprises obtaining a compound that binds to SEQ ID NO: 1.

In some embodiments, the compound is an ASO. In some embodiments, the ASO is an ASO as disclosed and as described herein.

Methods of assaying exon skipping are common. Non-limiting examples of such methods include, but are not limited to, PCR, qPCR, gene sequencing, northern-blot, dot-blot, in situ hybridization, or others all of which would be apparent to one of ordinary skill in the art.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value (±10%). For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

All scientific and technical terms used herein have meanings commonly used in the art, unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise”, or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting”.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); “Monoclonal Antibodies: Methods and Protocols”. Vincent Ossipow, Nicolas Fischer. Humana Press (2014); “Monoclonal Antibodies: Methods and Protocols”. Maher Albitar. Springer Science & Business Media (2007), all of which are incorporated by reference. Other general references are provided throughout this document.

### Example 1

### Stability of CCDC40 Lacking Exon 3 is Comparable to the WT CCDC40

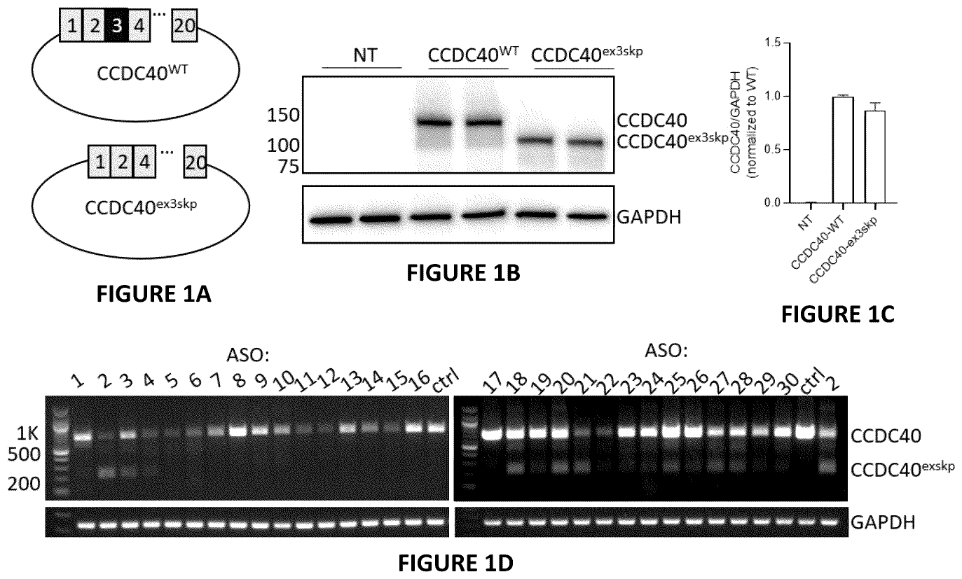

To assess whether CCDC40 with skipped exon 3 is expressed at the protein level, the inventors designed 2 plasmids: one that encodes the full cDNA sequence of wild type CCDC40 (CCDC40^{WT}), and a second one missing exon 3 (CCDC40^{ex3skp}) (FIG. 1A). The plasmids were ordered from VectorBuilder encoding the cDNA sequence of transcript NM_017950.4. HEK293T cells were transfected with 1 μg of each plasmid using FuGene HD transfection reagent, and protein lysates were prepared 48 hrs post-transfection in RIPA buffer with protease/phosphatase inhibitor cocktail. CCDC40 expression was analyzed by western blot using a specific anti-CCDC40 antibody (Proteintech #25049-1-AP) that detects the C-terminal region of CCDC40 (FIG. 1B). The protein expression of CCDC40^{ex3skp} appeared to be similar to CCDC40^{WT} suggesting that CCDC40^{ex3skp} is stable and not targeted for degradation.

### Example 2

### ASOs Induce Skipping of Exon 3

To identify ASOs capable of inducing CCDC40 exon 3 skipping, the inventors designed a mini-screen of 30 ASOs targeting CCDC40 exon 3 and an ASO control (FIG. 1F). The ASOs comprised 20 nucleotides with full phosphorothioate (PS) and 2′-O-methoxyethyl (2′-MOE) chemical modifications. The PS modification improves stability, increases nuclease resistance, and enhances transport over cell membranes. The 2′-MOE modification increases binding affinity to RNA, and further improves nuclease resistance. Importantly, when all the 20 nucleotides are modified with 2′-MOE, the ASO does not recruit RNase-H, therefore, it is suitable for exon-skipping experiments. A549 cells (lung epithelium) were transfected with 500 nM of the different ASOs using Lipofectamine 2000 transfection reagent. Forty-eight hours post-transfection RNA was extracted, reverse transcribed, and exon skipping was assessed by semi-quantitative PCR (FIG. 1D). Densitometry analysis of the first mini-screen (ASOs 1-16) revealed that ASOs 2, 3, and 4 (SEQ ID Nos: 5-7) induced skipping of exon 3 of CCDC40 compared to cells treated with a control ASO (FIG. 1E left graph), respectively. A second mini-screen (ASOs 17-30) was designed to increase the resolution around the binding site of ASO-2. Densitometry analysis revealed that most ASOs in this region induce skipping of exon 3 compared to a control ASO (FIG. 1E right graph).

### Example 3

### CCDC40 Lacking Exon 3 Co-Localizes with Microtubules

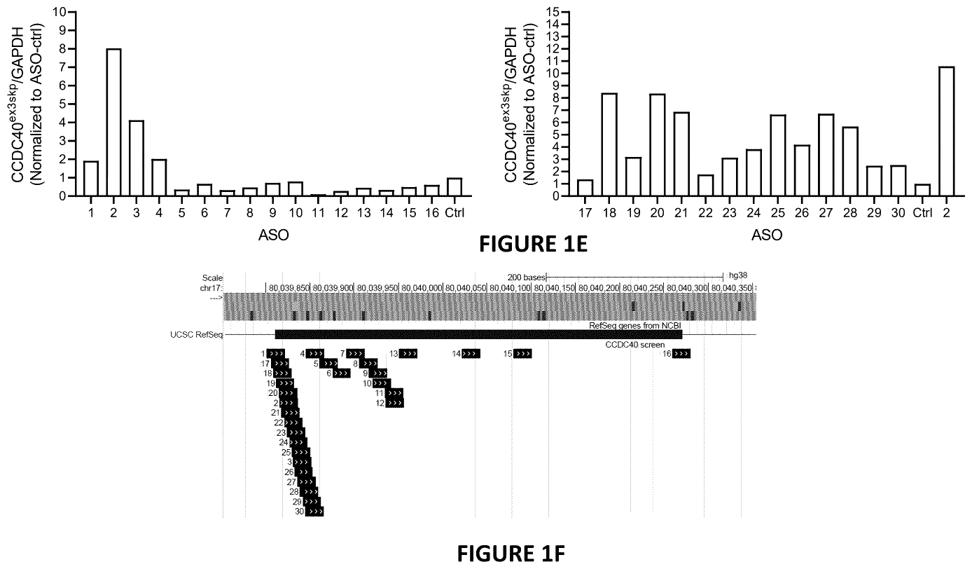

As a first step to evaluate functionality of CCDC40^{ex3skp}, the inventors set out to compare sub-cellular localization of CCDC40^{ex3skp} with CCDC40^{WT}-known to co-localize with the microtubules. To this end, the inventors evaluated co-localization by immunofluorescence in Hela cells following transient transfection of CCDC40^{ex3skp} and CCDC40^{WT} plasmids (48 hrs.). CCDC40 in both plasmids was tagged with Flag at the C-terminus and was detected with anti-Flag antibody (Sigma-Aldrich #F1804). Microtubule were detected using an anti-Tubulin antibody (Abcam #ab6046). The nuclei of cells were stained with Hoechst 33342. Images were taken with a confocal Leica microscope. Both CCDC40^{WT} and CCDC40^{ex3skp} appear similarly expressed and co-localized with microtubules, suggesting that the lack of exon 3 does not affect CCDC40 basic functional properties (FIG. 2).

### Example 4

### CCDC40 with Skipped Exon 3—Maintains a Physical Interaction with CCDC39

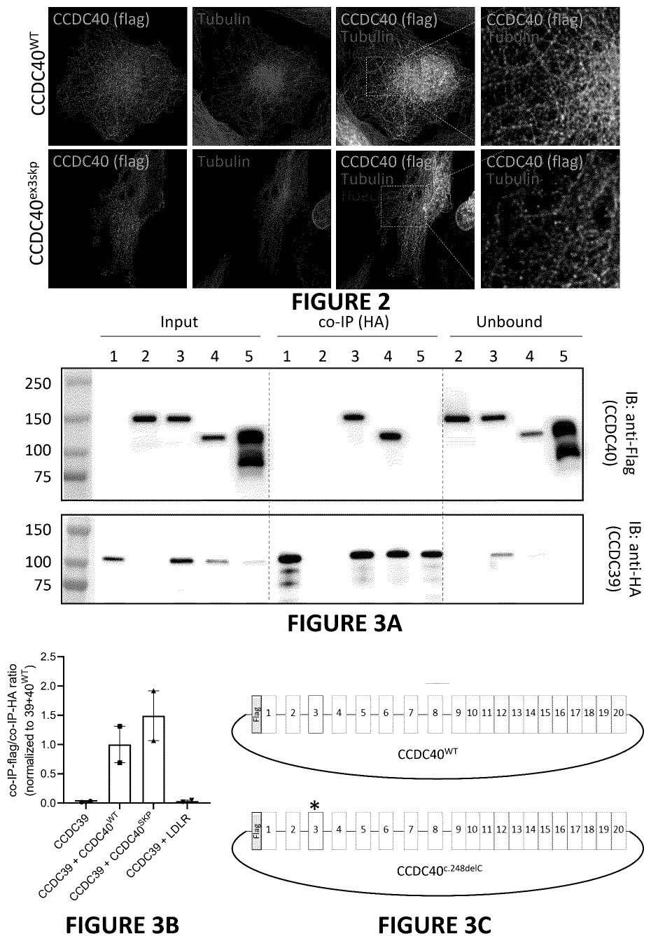

To further evaluate functionality of CCDC40^{ex3skp}, the inventors demonstrated a physical and functional relationship between WT and skipped versions of CCDC40 and its known interactor-CCDC39 (Merveille et al., Nat Genet (2011)). Structural studies have demonstrated that CCDC39 and CCDC40 form a complex important for guiding the placement of radial spokes along doublet microtubule that form motile cilia (Gui et al., Nat Struct Mol Biol (2021)). Through co-immunoprecipitation of plasmids encoding cDNA of CCDC39 and either CCDC40^{WT} or CCDC40^{ex3skp}, the inventors demonstrated a physical interaction between CCDC39 and CCDC40^{WT} and an interaction with similar intensity between CCDC39 and CCDC40^{ex3skp}. This strengthens the hypothesis that excluding exon 3 from CCDC40 does not abolish its interaction with CCDC39 (FIGS. 3A-3B). In this experiment, HEK293T cells were transfected with 1 μg of plasmid (for single transfection) or 0.5 μg each (for dual transfection) using FuGene HD transfection reagent. Protein lysates were prepared 48 hrs. post-transfection in RIPA IP buffer with protease/phosphatase inhibitor cocktail. The protein lysate was then subjected to immunoprecipitation with HA magnetic beads (ThermoFisher #88837) following the manufacturer's instructions. The amount of CCDC40 co-immunoprecipitated with CCDC39 was analyzed by western blot using anti-Flag antibody. Successful binding of the HA beads to CCDC39-HA was analyzed by western blot using anti-HA antibody. LDLR was used as a flag-tagged negative control as it is known not to interact with CCDC39. Next, the inventors demonstrated rescue of this interaction using ASOs on a mutated copy of the gene (FIGS. 3C-3D). To this end, the inventors designed CCDC40 plasmids with partial introns (300 bp from each end of the intron) between exons 1-9, after which the inventors included only cDNA (FIG. 3C). WT and mutant (c.248delC) CCDC40 plasmids with partial introns were co-immunoprecipitated with CCDC39 plasmids with or without ASO-2, which was previously demonstrated to induce the skipping of exon 3. The results show that ASO-2 not only has the ability to rescue the truncated mutant protein, but can also restore its interaction with CCDC39 (FIG. 3D).

### Example 5

### CCDC40 WT and Skipped Version Co-Localize with CCDC39 to the Microtubule

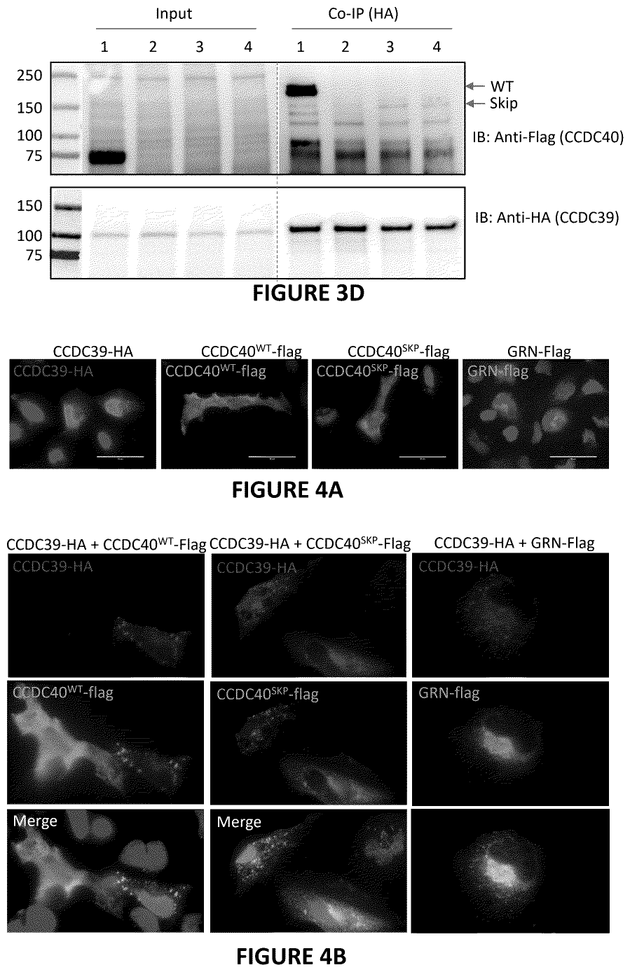

Further, through immunofluorescence in Hela cells following transient co-transfection of CCDC39 and CCDC40 plasmids, the inventors demonstrated that CCDC39 alters cellular localization of both CCDC40^{WT} and CCDC40^{ex3skp} in a similar manner (FIG. 4). First, CCDC40-flag, CCDC40^{ex3skp}-flag, CCDC39-HA, and GRN-flag (as a negative control as it is known not to interact with CCDC39) plasmids were singly transfected into Hela cells (FIG. 4A). Following 48 hrs., cells were fixated and immunostained with anti-Flag and anti-HA antibodies. The nuclei of the cells were stained with Hoechst 33342. Images were taken with an EVOS FL microscope. When singly transfected, CCDC40 and CCDC40^{ex3skp} appear uniformly dispersed across the microtubule in the cell body, whereas CCDC39 appears to be concentrated in small spots along the microtubule (FIG. 4A). Transfection of CCDC39 together with CCDC40 leads to the co-localization of the later with CCDC39 along the microtubule in small spots. This co-localization with CCDC39 was consistent for both the WT and skipped versions or forms of CCDC40, but did not seem to occur when co-transfecting cells with CCDC39 and an unrelated control gene (FIG. 4B).

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

What is claimed is:

1. A method for treating primary ciliary dyskinesia (PCD) in a subject in need thereof, comprising administering to said subject a therapeutically effective amount of at least one synthetic antisense oligonucleotide (ASO), wherein said ASO induces the skipping of exon 3 of the coiled-coil domain containing 40 (CCDC40) pre-mRNA, thereby treating PCD in the subject.

2. The method of claim 1, wherein said ASO comprises a backbone selected from the group consisting of: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

3. The method of claim 1 or 2, wherein said ASO comprises 14 to 25 bases.

4. The method of any one of claims 1 to 3, wherein said ASO has at least 75% complementarity to an equal-length portion of a nucleic acid sequence derived from the polynucleotide sequence:
(SEQ ID NO: 1)
GTGTCACCACCAGAGAAGGATGATGGCCAGAAAGGTGAAGAAGCT
GTCGGTAGCACAGAGCATCCTGAGGAAGTCACAACCCAAGCGGAA
GCTGCAATTGAAGAGGGGGAGGTGGAGACAGAAGGGGAAGCAGCA
GTGGAAGGGGAAGAGGAGGCTGTGTCCTATGGAGATGCTGAAAGC
GAAGAGGAATATTACTATACAGAAACTTCATCCCCGGAAGGGCAA
ATCAGTGCTGCAGATACGACTTACCCGTATTTCAGTCCTCCTCAG
GAACTGCCTGGAGAGGAGGCATACGATAGTGTTAGCGGGGAGGCT
GGTCTCCAAGGCTTCCAGCAAGAGGCCACCGGTCCACCAGAATCC
AGAGAAAGGAGGGTCACCTCCCCAGAGCCATCCCACGGAGTCTTA
GGCCCGTCGGAGCAAATGGGCCAGGTCACCTCTGGGCCAGCAGTG
GGCAGATTG.

5. The method of any one of claims 1 to 4, wherein said ASO has at least 75% complementarity to any one of: TGTCACCACCAGAGAAGGATGATGGCCAG (SEQ ID NO: 2), AGGATGATGGCCAGAAAGGTGAAGAAGC (SEQ ID NO: 3), and AAGGTGAAGAAGCTGTCGGTAGCACAGA (SEQ ID NO: 4).

6. The method of any one of claims 1 to 5, wherein said ASO comprises the nucleic acid sequence set forth in any one of: SED ID Nos: 5-7, 23-26, and 28-35.

7. The method of any one of claims 1 to 6, wherein said ASO comprises the nucleic acid sequence set forth in any one of: SED ID Nos: 5, 6, 23, 25, 26, and 30-33.

8. The method of any one of claims 1 to 7, wherein said ASO comprises the nucleic acid sequence set forth in any one of: SED ID Nos: 5, 23, 25, 26, 30, and 32.

9. The method of any one of claims 1 to 8, wherein said subject comprises at least one nonsense and/or frameshift mutation in exon 3 of CCDC40.

10. The method of claim 9, wherein said at least one mutation is c.248delC.

11. A composition comprising an ASO comprising 14 to 25 bases having at least 80% complementarity to a CCDC40 pre-mRNA and characterized by inducing splicing activity of exon 3 of said CCDC40 pre-mRNA.

12. The composition of claim 11, wherein said ASO comprises the nucleic acid sequence set forth in SED ID Nos: 5-7, 23-26, and 28-35.

13. The composition of claim 11 or 12, wherein said ASO comprises the nucleic acid sequence set forth in SED ID Nos: 5, 6, 23, 25, 26, and 30-33.

14. The composition of claim 11 or 12, wherein said ASO comprises the nucleic acid sequence set forth in SED ID Nos: 5, 23, 25, 26, 30, and 32.

15. The composition of any one of claims 11 to 14, wherein said ASO comprises a chemically modified backbone.

16. The composition of claim 15, wherein said chemically modified backbone comprises: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

17. The composition of any one of claims 11 to 16, further comprising a pharmaceutically acceptable carrier.

18. The composition of any one of claims 11 to 17, being an inhalation composition.

19. The composition of any one of claims 11 to 18, for use in the treatment of PCD in a subject in need thereof.

20. The composition of any one of claims 11 to 18, for use in inducing the skipping of exon 3 of the CCDC40 pre-mRNA in a subject in need thereof, or in a cell of said subject.

21. The composition for use of claim 20, wherein said subject is afflicted with PCD.
